(19) United States
(12) Patent Application Publication
(10) Pub. No.: US 2002/0132302 A1
(43) Pub. Date: Sep. 19, 2002

(54) TMOF receptor and uses thereof

(76) Inventor: Dov Borovsky, Vero Beach, FL (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/090,696

(22) Filed: Mar. 4, 2002

Related U.S. Application Data
(63) Continuation of application No. 09/201,568, filed on Nov. 30, 1998.
(60) Provisional application No. 60/102,230, filed on Sep. 29, 1998.

(51) Int. Cl. C07K 14/705; C07H 21/04; C12P 21/02; C12N 5/04; C12N 5/06

(52) U.S. Cl. 435/69.1; 435/325; 435/410; 435/320.1; 530/350; 536/23.5

(57) ABSTRACT

The subject invention provides Trypsin Modulating Oostatic Factor receptors and methods of identifying novel insect control agents.

### CROSS-REFERENCE TO A RELATED APPLICATION

This application is a continuation of copending application Ser. No. 09/201,568, filed Nov. 30, 1998. This application also claims the benefit of provisional patent application Serial No. 60/102,230, filed Sep. 29, 1998.

### BACKGROUND OF THE INVENTION

Mosquitoes and many agricultural insect pests digest their food using trypsin-like enzymes that are synthesized in the midgut epithelial cells. After feeding, a signal is sent to the gut epithelial cells to initiate trypsin biosynthesis. Trypsins are well characterized enzymes and their sequences and three dimensional conformations are known.

In the mosquito Aedes aegypti, an early trypsin that is found in the midgut of newly emerged females is replaced, following the blood meal, by the late trypsin that is synthesized in a very short time; a female mosquito weighs 2 mg and produces 4 to 6 μg trypsin within several hours after the blood meal. If trypsin were to be continually synthesized at this rate, female mosquitoes would spend all their energy on trypsin biosynthesis and would neither be able to mature their eggs nor find an oviposition site. To conserve energy, the mosquito regulates trypsin biosynthesis with a hormone named Trypsin Modulating Oostatic Factor (TMOF). TMOF is synthesized in the follicular epithelium of the ovary 2-30 hours after a blood meal and is released into the hemolymph, and binds to a specific receptor on the midgut epithelial cells signaling the termination of trypsin biosynthesis.

This regulatory mechanism is not unique for mosquitoes; fleshflies, fleas, sandflies, house flies, dogflies and other pests which may not feed on blood have a similar regulatory mechanism.

### BRIEF SUMMARY OF THE INVENTION

The subject invention pertains to materials and methods useful in the control of pests. The subject invention further provides materials and methods useful in the identification of novel pest control agents.

In one embodiment, the subject invention pertains to the identification of receptors for Trypsin Modulating Oostatic Factor (TMOF). A further aspect of the subject invention pertains to the identification of polynucleotide sequences which encode the TMOF receptor.

Specifically exemplified herein is the TMOF receptor from the mosquito, Aedes aegypti. Using the teachings provided herein, those skilled in the art can readily obtain and use TMOF receptors, and polynucleotides encoding these receptors, from other species.

The TMOF receptors of the subject invention are useful in identifying and purifying novel pest control agents which bind to the TMOF receptor. Thus, in one embodiment, the subject invention provides materials and methods for identifying novel pest control compounds.

The subject invention further pertains to pest control compounds which bind to a TMOF receptor. These pest control compounds can be used, as described herein, to control a broad range of pests. Specifically exemplified herein is the control of mosquitoes using pest control agents which bind to the TMOF receptor. Other biting pests such as flies, fleas, ticks, and lice can also be controlled using the pest control agents and methods of the subject invention.

The pest control agents of the subject invention can also be used to control pests of agricultural crops. These pests include, for example, coleopterans (beatles), lepidopterans (caterpillars), and mites. The compounds of the subject invention can also be used to control household pests including, but not limited to, ants and cockroaches.

The subject invention provides pest control compositions wherein the pest control agents are formulated for application to the target pests, or their situs. In a specific embodiment, recombinant hosts, which express a pest control agent are provided by the subject invention. The recombinant host may be, for example, procaryotic or eucaryotic. In a specific example, yeast or algae are transformed to express a pest control compound of the subject invention. The transformed hosts are then applied to water areas where mosquito larvae will ingest the transformed host resulting in control of the mosquitoes by the pest control agent.

In a preferred embodiment for the control of agricultural pests, the subject invention provides transformed plants which express a pest control compound. Pest control is achieved when the pest ingests the transformed plant material.

### BRIEF DESCRIPTION OF THE SEQUENCES

SEQ ID NO. 1 is a polynucleotide sequence encoding a portion of a TMOF receptor.

SEQ ID NO. 2 is the amino acid sequence encoded by the polynucleotide sequence of SEQ ID NO. 1.

### DETAILED DISCLOSURE OF THE INVENTION

The subject invention is directed to Trypsin Modulating Oostatic Factor (TMOF) receptors, polynucleotides which encode TMOF receptors, and uses thereof. One aspect of the subject invention pertains to polynucleotides useful as probes to identify TMOF receptor genes from a broad spectrum of species. A related aspect of the subject invention pertains to the identification of polynucleotide sequences which encode polypeptides which exhibit TMOF receptor activity.

In one embodiment, the materials and methods of the subject invention may be used to identify novel pest control compounds. The subject invention further pertains to novel pest control agents which bind to the TMOF receptor. By binding to the TMOF receptor these agents kill or otherwise control pests. The subject invention further concerns the use of these novel pest control agents to control agricultural pests as well as pests of humans and animals.

Yet another aspect of the subject invention pertains to cells transformed with a polynucleotide which encodes a polypeptide exhibiting TMOF receptor activity.

In a specific embodiment, the subject invention is directed to a method of identifying pest control agents. Specifically, TMOF receptors can be used to identify compounds which bind to these receptors thereby causing deleterious effects to a target pest having a TMOF receptor.

As described more fully herein, the TMOF receptors of the subject invention can be used in several ways to identify novel pest control agents. One such method comprises transforming a cell with a polynucleotide encoding a polypeptide having TMOF receptor activity; expressing the polynucleotide such that said polypeptide is positioned on the cell membrane of the cell; and testing the ability of a compound of interest to bind to the TMOF receptor polypeptide.

The term “TMOF receptor activity,” as used herein, means an ability to associate with TMOF, or fragments or mutants thereof. In a preferred embodiment of the subject invention, this association with the TMOF receptor is of a nature such that, when the compound is applied to a pest having a TMOF receptor, control of the pest is achieved. As used herein, reference to a “TMOF receptor” means a molecule which has TMOF receptor activity.

As used herein, reference to “isolated” polynucleotides and/or “purified” toxins refers to these molecules when they are not associated with the other molecules with which they would be found in nature. Thus, reference to “isolated” and/or “purified” signifies the involvement of the “hand of man” as described herein.

TMOF Receptors and Polynucleotides

In one embodiment, the subject invention is directed to polypeptide molecules having TMOF receptor activity. Specifically exemplified herein is a TMOF receptor comprising the amino acid sequence shown in SEQ ID NO. 2. Preferably, the polypeptide is encoded by a complete cDNA sequence of a TMOF receptor gene, or fragments or mutants thereof which encode polypeptides having TMOF receptor activity. In a specific embodiment, the TMOF receptor is encoded by a polynucleotide sequence comprising the coding sequence (nucleotides 1-186) shown in SEQ ID NO. 1 or other polynucleotide sequence with codons encoding the amino acid sequence of SEQ ID NO. 2.

The polypeptides of the subject invention can be purified using standard protein purification procedures well known in the art. The sequences of polypeptides of the subject invention can be derived from their corresponding polynucleotide sequences or elucidated using peptide sequencing procedures known in the art.

Isolated polypeptides of the subject invention can be used to produce antibodies according to known techniques. These antibodies may be monoclonal or polyclonal. These antibodies can be used to screen an expression library to identify other clones expressing polypeptides having TMOF receptor activity. Alternatively, these antibodies may be used to identify TMOF receptors from their natural material such as, for example, mosquito gut material.

A specific TMOF receptor sequence is exemplified herein. This sequence is merely exemplary of the receptors of the subject invention; the subject invention comprises variant or equivalent receptors (and nucleotide sequences coding for equivalent receptors) having the same or similar TMOF receptor activity as the exemplified receptor. Equivalent receptors will typically have amino acid homology with the exemplified receptor. This amino acid identity will typically be greater than 60%, preferably be greater than 75%, more preferably greater than 80%, more preferably greater than 90%, and can be greater than 95%. These identities are as determined using standard alignment techniques. The amino acid homology will be highest in critical regions of the receptor which account for biological activity or are involved in the determination of three-dimensional configuration which ultimately is responsible for the biological activity. In this regard, certain amino acid substitutions are acceptable and can be expected if these substitutions are in regions which are not critical to activity or are conservative amino acid substitutions which do not affect the three-dimensional configuration of the molecule. For example, amino acids may be placed in the following classes: non-polar, uncharged polar, basic, and acidic. Conservative substitutions whereby an amino acid of one class is replaced with another amino acid of the same type fall within the scope of the subject invention so long as the substitution does not materially alter the biological activity of the compound. Table 1 provides a listing of examples of amino acids belonging to each class.

TABLE 1

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

In some instances, non-conservative substitutions can also be made. The critical factor is that these substitutions must not significantly detract from the biological activity of the receptor.

Another embodiment of the subject invention is directed to polynucleotide molecules useful as probes to identify and/or characterize polynucleotides encoding polypeptides having TMOF receptor activity. These polynucleotide sequences may be RNA or DNA. In a specific embodiment, SEQ ID NO. 1, or its complementary sequence, or fragments or mutants thereof, can be used as a probe to identify polynucleotides which encode TMOF receptors.

It is well known that DNA possesses a fundamental property called base complementarity. In nature, DNA ordinarily exists in the form of pairs of anti-parallel strands, the bases on each strand projecting from that strand toward the opposite strand. The base adenine (A) on one strand will always be opposed to the base thymine (T) on the other strand, and the base guanine (G) will be opposed to the base cytosine (C). The bases are held in apposition by their ability to hydrogen bond in this specific way. Though each individual bond is relatively weak, the net effect of many adjacent hydrogen bonded bases, together with base stacking effects, is a stable joining of the two complementary strands. These bonds can be broken by treatments such as high pH or high temperature, and these conditions result in the dissociation, or “denaturation,” of the two strands. If the DNA is then placed in conditions which make hydrogen bonding of the bases thermodynamically favorable, the DNA strands will anneal, or “hybridize,” and reform the original double stranded DNA. If carried out under appropriate conditions, this hybridization can be highly specific. That is, only strands with a high degree of base complementarity will be able to form stable double stranded structures. The relationship of the specificity of hybridization to reaction conditions is well known. Thus, hybridization may be used to test whether two pieces of DNA are complementary in their base sequences. It is this hybridization mechanism which facilitates the use of probes of the subject invention to readily detect and characterize DNA sequences of interest.

The probes may be RNA or DNA. The probe will normally have at least about 10 bases, more usually at least about 17 bases, and may have up to about 100 bases or more. Longer probes can readily be utilized, and such probes can be, for example, several kilobases in length. The probe need not have perfect complementarity to the sequence to which it hybridizes. The probes may be labeled utilizing techniques which are well known to those skilled in this art.

The use of polynucleotide probes is well known to those skilled in the art. In one specific example, a cDNA library for mosquito gut cells can be created by routine means, and DNA of interest isolated therefrom. Polynucleotides of the subject invention can be used to hybridize with DNA fragments of the constructed cDNA-library, allowing identification of and selection (or “probing out”) of the genes of interest, i.e., those nucleotide sequences which hybridize with the probes of the subject invention and encode polypeptides having TMOF receptor activity. The isolation of these genes can be performed by a person skilled in the art, having the benefit of the instant disclosure, using techniques which are well-known in the molecular biology art.

Thus, it is possible, without the aid of biological analysis, to identify polynucleotide sequences encoding TMOF receptors. Such a probe analysis provides a rapid method for identifying genes encoding TMOF receptors from a wide variety of hosts. Accordingly, another embodiment of the subject invention is an isolated polynucleotide molecule which encodes a polypeptide having TMOF receptor activity. The isolated genes can be inserted into appropriate vehicles which can then be used to transform a suitable host. In addition, these genes can be sequenced by standard nucleic acid sequencing procedures to provide specific information about the base composition of the genes encoding the subject polypeptides.

One hybridization procedure useful according to the subject invention typically includes the initial steps of isolating the DNA sample of interest and purifying it chemically. The DNA sample can be cut into pieces with an appropriate restriction enzyme. The pieces can be separated by size through electrophoresis in a gel, usually agarose or acrylamide. The pieces of interest can be transferred to an immobilizing membrane.

The particular hybridization technique is not essential to the subject invention. As improvements are made in hybridization techniques, they can be readily applied.

The probe and sample can then be combined in a hybridization buffer solution and held at an appropriate temperature until annealing occurs. Thereafter, the membrane is washed free of extraneous materials, leaving the sample and bound probe molecules typically detected and quantified by autoradiography and/or liquid scintillation counting or other techniques (e.g. fluorescence, enzyme assay, immunoassay or combinations thereof). As is well known in the art, if the probe molecule and nucleic acid sample hybridize by forming a strong non-covalent bond between the two molecules, it can be reasonably assumed that the probe and sample are essentially identical. The probe's detectable label provides a means for determining in a known manner whether hybridization has occurred.

In the use of the nucleotide segments as probes, the particular probe is labeled with any suitable label known to those skilled in the art, including radioactive and non-radioactive labels. Typical radioactive labels include ³²P, ³⁵S, or the like. Non-radioactive labels include, for example, ligands such as biotin or thyroxine, as well as enzymes such as hydrolases or perixodases, or the various chemiluminescers such as luciferin, or fluorescent compounds like fluorescein and its derivatives. The probes may be made inherently fluorescent as described in International Application No. WO 93/16094.

Various degrees of stringency of hybridization can be employed. The more severe the conditions, the greater the complementarity that is required for duplex formation. Severity of conditions can be controlled by temperature, probe concentration, probe length, ionic strength, time, and the like. Preferably, hybridization is conducted under moderate to high stringency conditions by techniques well known in the art, as described, for example, in Keller, G. H., M. M. Manak (1987) DNA Probes, Stockton Press, New York, N.Y., pp. 169-170.

Examples of various stringency conditions are provided herein. Hybridization of immobilized DNA on Southern blots with ³²P-labeled gene-specific probes can be performed by standard methods (Maniatis et al.). In general, hybridization and subsequent washes can be carried out under moderate to high stringency conditions that allow for detection of target sequences with homology to the exemplified polynucleotide sequence. For double-stranded DNA gene probes, hybridization can be carried out overnight at 20-25° C. below the melting temperature (Tm) of the DNA hybrid in 6× SSPE, 5× Denhardt's solution, 0.1% SDS, 0.1 mg/ml denatured DNA. The melting temperature is described by the following formula (Beltz, G. A., K. A. Jacobs, T. H. Eickbush, P. T. Cherbas, and F. C. Kafatos [1983] Methods of Enzymology, R. Wu, L. Grossman and K. Moldave [eds.] Academic Press, New York 100:266-285):

Tm=81.5° C.+16.6 Log[Na⁺]+0.41(%G+C)−0.61(%formamide)−600/length of duplex in base pairs.

Washes are typically carried out as follows:

(1) Twice at room temperature for 15 minutes in 1× SSPE, 0.1% SDS (low stringency wash).

(2) Once at Tm-20° C. for 15 minutes in 0.2× SSPE, 0.1% SDS (moderate stringency wash).

For oligonucleotide probes, hybridization can be carried out overnight at 10-20° C. below the melting temperature (Tm) of the hybrid in 6× SSPE, 5× Denhardt's solution, 0.1% SDS, 0.1 mg/ml denatured DNA. Tm for oligonucleotide probes can be determined by the following formula:

Tm(° C.)=2(number T/A base pairs)+4(number G/C base pairs)

(Suggs, S. V., T. Miyake, E. H. Kawashime, M. J. Johnson, K. Itakura, and R. B. Wallace [1981] ICN-UCLA Symp. Dev. Biol. Using Purified Genes, D. D. Brown [ed.], Academic Press, New York, 23:683-693).

Washes can be carried out as follows:

(1) Twice at room temperature for 15 minutes 1× SSPE, 0.1% SDS (low stringency wash).

(2) Once at the hybridization temperature for 15 minutes in 1× SSPE, 0.1% SDS (moderate stringency wash).

In general, salt and/or temperature can be altered to change stringency. With a labeled DNA fragment >70 or so bases in length, the following conditions can be used:

Low: 1 or 2× SSPE, room temperature

Low: 1 or 2× SSPE, 42° C.

Moderate: 0.2× or 1× SSPE, 65° C.

High: 0.1× SSPE, 65° C.

Duplex formation and stability depend on substantial complementarity between the two strands of a hybrid, and, as noted above, a certain degree of mismatch can be tolerated. Therefore, the probe sequences of the subject invention include mutations (both single and multiple), deletions, insertions of the described sequences, and combinations thereof, wherein said mutations, insertions and deletions permit formation of stable hybrids with the target polynucleotide of interest. Mutations, insertions, and deletions can be produced in a given polynucleotide sequence in many ways, and these methods are known to an ordinarily skilled artisan. Other methods may become known in the future.

Thus, mutational, insertional, and deletional variants of the disclosed nucleotide sequence can be readily prepared by methods which are well known to those skilled in the art. As used herein, substantial sequence homology refers to homology which is sufficient to enable the variant probe to function in the same capacity as the original probe. Preferably, this homology is greater than 60%; more preferably, this homology is greater than 75%; and most preferably, this homology is greater than 90%.

PCR Technology.

Polymerase Chain Reaction (PCR) is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki, Randall K., Stephen Scharf, Fred Faloona, Kary B. Mullis, Glenn T. Horn, Henry A. Erlich, Norman Arnheim

“Enzymatic Amplification of β-Globin Genomic Sequences and Restriction Site Analysis for Diagnosis of Sickle Cell Anemia,” Science 230:1350-1354.). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Since the extension product of each primer can serve as a template for the other primer, each cycle essentially doubles the amount of DNA fragment produced in the previous cycle. This results in the exponential accumulation of the specific target fragment, up to several million-fold in a few hours. By using a thermostable DNA polymerase such as Taq polymerase, which is isolated from the thermophilic bacterium Thermus aquaticus, the amplification process can be completely automated. Other enzymes which can be used are known to those skilled in the art.

PCR primers can be designed from the DNA sequence of the subject invention. In performing PCR amplification, a certain degree of mismatch can be tolerated between primer and template. Therefore, mutations, deletions, and insertions (especially additions of nucleotides to the 5′ end) of the exemplified sequence fall within the scope of the subject invention. These PCR primers can be used to amplify genes of interest from a sample. Thus, this is another method by which polynucleotide sequences encoding TMOF receptors can be identified and characterized.

Identification of Pest Control Compounds

The TMOF receptors of the subject invention can, advantageously, be used to identify pest control compounds. These compounds are those which bind to, or otherwise associate with, the TMOF receptor in a way in which inhibits natural function of the TMOF receptor thereby inhibiting or killing a pest. A person skilled in the art, having the benefit of the instant disclosure, can utilize the TMOF receptors described herein to identify novel pest control compounds. In one embodiment, the TMOF receptor can be purified from its natural sources using, for example, antibodies to the TMOF receptor to obtain the purified protein. This purified protein can then be used to identify compounds which bind to the receptor. Compounds thus identified can then be further evaluated using, for example, appropriate bioassays to confirm and/or characterize the pest control activity of the compound.

As an alternative to purifying TMOF receptors from their natural material, recombinant TMOF receptor protein can be expressed in an appropriate recombinant host which has been transformed with a polynucleotide sequence encoding the TMOF receptor. The polynucleotide sequence used to transform the appropriate host may comprise, for example, the polynucleotide coding sequence disclosed in SEQ ID NO. 1. The host may be transformed so as to express the TMOF receptor at the cell surface or, alternatively, the TMOF receptor may be retained intracellularly or secreted into the surrounding media. In any case, the expressed TMOF receptor may be isolated from the recombinant host using techniques known to those skilled in the art. The recombinant purified protein can then be used as described above to identify compounds which bind to the receptor. As an alternative embodiment, the receptor expressed at the surface of the recombinant cell can be used in conjunction with the whole cell to identify compounds which bind to the receptor.

In a specific embodiment, the subject invention provides a method of screening compounds to identify trypsin synthesis-inhibiting compounds. A preferred method involves exposing the compounds in a competitive binding assay to a TMOF receptor. The TMOF receptor may comprise the amino acid sequence of SEQ ID NO. 2.

In a specific embodiment, cDNA encoding polypeptides having TMOF receptor activity can be isolated and then inserted into a suitable cloning vector which is introduced into an appropriate host. Depending on the contemplated host, the vector may include various regulatory and other regions, usually including an origin of replication, and one or more promoter regions and markers for the selection of transformants. In general, the vectors provide regulatory signals for expression, amplification, and for a regulated response to a variety of conditions and reagents.

Various markers may be employed for the selection of transformants, including biocide resistance, particularly to antibiotics such as ampicillin, tetracycline, trimethoprim, chloramphenicol, and penicillin; toxins, such as colicin; and heavy metals, such as mercuric salts. Alternatively, complementation providing an essential nutrient to an auxotrophic host may be employed.

In another embodiment, the subject invention is directed to a cell transformed with a polynucleotide encoding a polypeptide having TMOF receptor activity. Hosts which may be employed according to techniques well known in the art for the production of the polypeptides of the present invention include unicellular microorganisms, such as prokaryotes, i.e., bacteria; and eukaryotes, such as fungi, including yeasts, algae, protozoa, molds, and the like, as well as plant cells, both in culture or in planta, and animal cells. Specific bacteria which are susceptible to transformation include members of the Enterobacteriaceae, such as strains of Escherichia coli; Salmonella; Bacillaceae, such as Bacillus subtilis; Pseudomonas; Pneumococcus; Streptococcus; Haemophilus influenzae, and yeasts such as Saccharomyces, among others.

The polynucleotide sequences of the subject invention can be introduced directly into the genome of the transformable host cell or can first be incorporated into a vector which is then introduced into the host. Exemplary methods of incorporation include transduction by recombinant phage or cosmids, transfection where specially treated host bacterial cells can be caused to take up naked phage chromosomes, and transformation by calcium precipitation. These methods are well known in the art. Exemplary vectors include plasmids, cosmids, and phages.

It is well known in the art that when synthesizing a gene for improved expression in a host cell it is desirable to design the gene such that its frequency of codon usage approaches the frequency of preferred codon usage of the host cell. For purposes of the subject invention, “frequency of preferred codon usage” refers to the preference exhibited by a specific host cell in usage of nucleotide codons to specify a given amino acid. To determine the frequency of usage of a particular codon in a gene, the number of occurrences of that codon in the gene is divided by the total number of occurrences of all codons specifying the same amino acid in the gene. Similarly, the frequency of preferred codon usage exhibited by a host cell can be calculated by averaging frequency of preferred codon usage in a large number of genes expressed by the host cell. It is preferable that this analysis be limited to genes that are highly expressed by the host cell.

Thus, in one embodiment of the subject invention, bacteria, plants, or other cells can be genetically engineered, e.g., transformed with genes from mosquitoes or other pests to attain desired expression levels of the subject proteins. To provide genes having enhanced expression, the DNA sequence of the gene can be modified to comprise codons preferred by highly expressed genes to attain an A+T content in nucleotide base composition which is substantially that found in the transformed host cell. It is also preferable to form an initiation sequence optimal for the host cell, and to eliminate sequences that cause destabilization, inappropriate polyadenylation, degradation and termination of RNA and to avoid sequences that constitute secondary structure hairpins and RNA splice sites. For example, in synthetic genes, the codons used to specify a given amino acid can be selected with regard to the distribution frequency of codon usage employed in highly expressed genes in the host cell to specify that amino acid. As is appreciated by those skilled in the art, the distribution frequency of codon usage utilized in the synthetic gene is a determinant of the level of expression.

Assembly of the polynucleotide sequences of this invention can be performed using standard technology known in the art. A structural gene designed for enhanced expression in a host cell can be enzymatically assembled within a DNA vector from chemically synthesized oligonucleotide duplex segments. The gene can then be introduced into the host cell and expressed by means known to the art. Preferably, the protein produced upon expression of the synthetic gene is functionally equivalent to a native protein. According to the subject invention, “functionally equivalent” refers to retention of function such as, for example, TMOF receptor activity and/or pest control activity. A synthetic gene product which has at least one property relating to its activity or function, which is the same or similar to a natural protein is considered functionally equivalent thereto.

The nucleotide sequences of the subject invention can be truncated such that certain of the resulting fragments of the original full-length sequence can retain the desired characteristics of the full-length sequence. A wide variety of restriction enzymes are well known by ordinarily skilled artisans which are suitable for generating fragments from larger nucleic acid molecules. For example, it is also well known that Bal31 exonuclease can be conveniently used for time-controlled limited digestion of DNA. See, for example, Maniatis et al. (1982) Molecular Cloning: A Laboratory Manual, Cold Spring Harbor Laboratory, New York, pages 135-139. See also Wei et al. (1983) J. Biol. Chem. 258:13006 13512. By use of Bal31 exonuclease (commonly referred to as “erase-a-base” procedures) the ordinarily skilled artisan can remove nucleotides from either or both ends of the subject nucleic acids to generate a wide spectrum of fragments which are functionally equivalent to the subject nucleic acids. Labeling procedures are also well known, and the ordinarily skilled artisan would be able to routinely test or screen the labeled generated fragments for their hybridization characteristics for determining the utility of the fragments as probes.

In another embodiment, TMOF receptors of the subject invention can be applied to a chip or other suitable substrate to facilitate high through put screening of potential pest control compounds.

Once compounds are identified which bind to the TMOF receptor, their pesticidal activity can be confirmed and/or characterized using bioassays known to those skilled in the art. The pesticide compounds of the subject invention can have activity against a variety of pests. These pests include agricultural pests which attack plants as well as pests of animals which attack humans, agricultural animals, and/or domestic animals.

Use of Novel Pest Control Compounds

The plant pests which can be controlled by the compounds of the subject invention include those that belong to the orders colepterans, lepidopterans, hemiptera and thysanoptera. These insect pests all belong to the arthropod phylum. Other insects which can be controlled according to the subject invention include members of the orders diptera, siphonaptera, hymenoptera and phthiraptera. Other arthropod pests which can be controlled by the compounds of the subject invention include those in the arachnid family such as ticks, mites, and spiders.

The use of the compounds of the subject invention to control pests can be accomplished readily by those skilled in the art having the benefit of the instant disclosure. For example, the control compounds may be encapsulated, included in a granular form, solubilized in water or other appropriate solvent, powdered, and included into any appropriate formulation for direct application to the pest. In a preferred embodiment for the control of plant pests, plants may be genetically transformed to express the pest control compound such that a pest feeding upon the plant will ingest the control compound and thereby be controlled.

Furthermore, chimeric toxins may be used according to the subject invention. Methods have been developed for making useful chimeric toxins by combining portions of proteins. The portions which are combined need not, themselves, be pesticidal so long as the combination of portions creates a chimeric protein which is pesticidal. The chimeric toxins may include portions from toxins which do not necessarily act upon the TMOF receptor including, for example, toxins from Bacillus thuringiensis (B.t.). B.t. toxins and their various toxin domains are well known to those skilled in the art.

With the teachings provided herein, one skilled in the art could readily produce and use the various toxins and polynucleotide sequences described herein.

The polynucleotide sequences and toxins useful according to the subject invention include not only the full length sequences but also fragments of these sequences, variants, mutants, and fusion proteins which retain the characteristic pesticidal activity of the toxins specifically exemplified herein. As used herein, the terms “variants” or “variations” of genes refer to nucleotide sequences which encode the same toxins or which encode equivalent toxins having pesticidal activity. As used herein, the term “equivalent toxins” refers to toxins having the same or essentially the same biological activity against the target pests as the exemplified toxins.

Variations of genes may be readily constructed using standard techniques for making point mutations. Also, fragments of these genes can be made using commercially available exonucleases or endonucleases according to standard procedures. For example, enzymes such as Bal31 or site-directed mutagenesis can be used to systematically cut off nucleotides from the ends of these genes. Also, genes which encode active fragments may be obtained using a variety of restriction enzymes. Proteases may be used to directly obtain active fragments of these toxins.

Recombinant Hosts.

Polynucleotide sequences encoding pest control compounds (toxins) can be introduced into a wide variety of microbial or plant hosts. In the case of toxins, expression of the toxin gene results, directly or indirectly, in the production and maintenance of the pesticide. With suitable microbial hosts, e.g., yeast, chlorella, the microbes can be applied to the situs of the pest, where they will proliferate and be ingested. The result is a control of the pest. Alternatively, the microbe hosting the toxin gene can be killed and treated under conditions that prolong the activity of the toxin and stabilize the cell. The treated cell, which retains the toxic activity, then can be applied to the environment of the target pest.

Where the toxin gene is introduced via a suitable vector into a microbial host, and said host is applied to the environment in a living state, it is essential that certain host microbes be used. Microorganism hosts are selected which are known to occupy the “phytosphere” (phylloplane, phyllosphere, rhizosphere, and/or rhizoplane) of one or more crops of interest or the situs where the pest proliferates. These microorganisms are selected so as to be capable of successfully competing in the particular environment (crop and other insect habitats) with the wild-type organisms, provide for stable maintenance and expression of the gene expressing the polypeptide pesticide, and, desirably, provide for improved protection of the pesticide from environmental degradation and inactivation.

A large number of microorganisms are known to inhabit the phylloplane (the surface of the plant leaves) and/or the rhizosphere (the soil surrounding plant roots) of a wide variety of important crops. These microorganisms include bacteria, algae, and fungi. Of particular interest are microorganisms, such as bacteria, e.g., genera Pseudomonas, Erwinia, Serratia, Klebsiella, Xanthomonas, Streptomyces, Rhizobium, Rhodopseudomonas, Methylophilius, Agrobacterium, Acetobacter, Lactobacillus, Arthrobacter, Azotobacter, Leuconostoc, and Alcaligenes; fungi, particularly yeast, e.g., genera Saccharomyces, Cryptococcus, Kluyveromyces, Sporobolomyces, Rhodotorula, and Aureobasidium. Of particular interest are such phytosphere bacterial species as Pseudomonas syringae, Pseudomonas fluorescens, Serratia marcescens, Acetobacter xylinum, Agrobacterium tumefaciens, Rhodopseudomonas spheroides, Xanthomonas campestris, Rhizobium melioti, Alcaligenes entrophus, and Azotobacter vinlandii; and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces roseus, S. odorus, Kluyveromyces veronae, and Aureobasidium pollulans. Of particular interest are the pigmented microorganisms.

A wide variety of ways are available for introducing a polynucleotide sequence encoding a toxin into a microorganism host under conditions which allow for stable maintenance and expression of the gene. These methods are well known to those skilled in the art and are described, for example, in U.S. Pat. No. 5,135,867, which is incorporated herein by reference.

Synthetic genes which are functionally equivalent to the toxins of the subject invention can also be used to transform hosts. Methods for the production of synthetic genes can be found in, for example, U.S. Pat. No. 5,380,831.

Treatment of Cells.

Recombinant cells expressing a pest control compound can be treated to prolong the toxin activity and stabilize the cell. The pesticide microcapsule that is formed comprises the toxin within a cellular structure that has been stabilized and will protect the toxin when the microcapsule is applied to the environment of the target pest. Suitable host cells may include either prokaryotes or eukaryotes. As hosts, of particular interest will be the prokaryotes and the lower eukaryotes, such as algae and fungi. The cell will usually be intact and be substantially in the proliferative form when treated, rather than in a spore form.

Treatment of the microbial cell, e.g., a microbe containing the toxin gene, can be by chemical or physical means, or by a combination of chemical and/or physical means, so long as the technique does not deleteriously affect the properties of the toxin, nor diminish the cellular capability of protecting the toxin. Methods for treatment of microbial cells are disclosed in U.S. Pat. Nos. 4,695,455 and 4,695,462, which are incorporated herein by reference.

Methods and Formulations for Control of Pests.

Control of pests using the pest control compounds of the subject invention can be accomplished by a variety of methods known to those skilled in the art. These methods include, for example, the application of recombinant microbes to the pests (or their locations), and the transformation of plants with genes which encode the pesticidal toxins of the subject invention. Transformations can be made by those skilled in the art using standard techniques. Materials necessary for these transformations are disclosed herein or are otherwise readily available to the skilled artisan.

Formulated bait granules containing an attractant and the toxins, or recombinant microbes comprising toxin-encoding polynucleotide sequences, can be applied to the soil. Formulated product can also be applied as a seed-coating or root treatment or total plant treatment at later stages of the crop cycle. Plant and soil treatments may be employed as wettable powders, granules or dusts, by mixing with various inert materials, such as inorganic minerals (phyllosilicates, carbonates, sulfates, phosphates, and the like) or botanical materials (powdered corncobs, rice hulls, walnut shells, and the like). The formulations may include spreader-sticker adjuvants, stabilizing agents, other pesticidal additives, or surfactants. Liquid formulations may be aqueous-based or non-aqueous and employed as foams, gels, suspensions, emulsifiable concentrates, or the like. The ingredients may include Theological agents, surfactants, emulsifiers, dispersants, or polymers.

As would be appreciated by a person skilled in the art, the pesticidal concentration will vary widely depending upon the nature of the particular formulation, particularly whether it is a concentrate or to be used directly. The pesticide will be present in at least about 0.01% by weight and may be 100% by weight. The dry formulations will have from about 1-95% by weight of the pesticide while the liquid formulations will generally be from about 1-60% by weight of the solids in the liquid phase. The formulations that contain cells will generally have from about 10² to about 10⁴ cells/mg. These formulations will be administered at about 50 mg (liquid or dry) to 1 kg or more per hectare.

The formulations can be applied to the environment of the pest, e.g., soil and foliage, by spraying, dusting, sprinkling, or the like.

All of the U.S. patents cited herein are hereby incorporated by reference.

Following are examples which illustrate procedures for practicing the invention. These examples should not be construed as limiting. All percentages are by weight and all solvent mixture proportions are by volume unless otherwise noted.

### EXAMPLE 1

### Assay for TMOF-Like Ligands

Cells are transformed with a polynucleotide sequence encoding a polypeptide having TMOF receptor activity such that the polynucleotide is expressed and positioned on the cell membrane. The transformed cells are grown in microtiter plates and immobilized to the bottom of the plate wells. TMOF labeled with a fluorescence tag is added in the presence of a control agent of interest and allowed to bind to the receptor. The cells are then washed to remove unbound TMOF. The fluorescence is then measured to test the ability of the control agent of interest to compete for receptor binding.

### EXAMPLE 2

### Production of Purified Receptor Protein

Polynucleotides encoding TMOF receptors are transformed into yeast cells. Preferably the polynucleotide is incorporated into the yeast genome such that the yeast produces copious amounts of receptor protein. The yeast cells are homogenized and the receptor protein is isolated using protein purification techniques known in the art.

### EXAMPLE 3

### Assay Using Purified Protein

The purified protein produced in Example 2 is immobilized on the wells of microtiter plates. Fluorescence labeled TMOF is added to the wells in the presence of a control agent of interest. The competitive ability of the control agent is measured as described in Example 1.

### EXAMPLE 4

### Biological Activity of Compounds Which Bind to TMOF Receptors

Control agents which bind with TMOF receptors as described in, for example, Examples 1 and 3 can be tested to confirm and characterize pest control activity. Many bioassays are known to those skilled in the art for the purpose of evaluating pesticidal activity. Assays for evaluating mosquito control activity are known to those skilled in the art and are described in, for example, U.S. Pat. No. 5,436,002. Bioassays for evaluating the pest control activity against other targets are also known to those skilled in the art and are described in, for example, U.S. Pat. Nos. 5,596,071; 5,188,960; and 5,366,892.

Control agents which bind with TMOF receptors as described in, for example, Examples 1 and 3 can be tested to confirm and characterize pest control activity. Many bioassays are known to those skilled in the art for the purpose of evaluating pesticidal activity. Assays for evaluating mosquito control activity are known to those skilled in the art and are described in, for example, U.S. Pat. No. 5,436,002. Bioassays for evaluating the pest control activity against other targets are also known to those skilled in the art and are described in, for example, U.S. Pat. Nos. 5,596,071; 5,188,960; and 5,366,892.

### EXAMPLE 5

### Bioassays for Activity against Lepidopteron and Coleopterans

Biological activity of the control compounds of the subject invention can be confirmed using standard bioassay procedures. One such assay is the budworm-bollworm (Heliothis virescens [Fabricius] and Helicoverpa zea [Boddie]) assay. Lepidoptera bioassays can be conducted with either surface application to artificial insect diet or diet incorporation of samples. All Lepidopteran insects can be tested from the neonate stage to the second instar. All assays can be conducted with either toasted soy flour artificial diet or black cutworm artificial diet (BioServ, Frenchtown, N.J.).

Diet incorporation can be conducted by mixing the samples with artificial diet at a rate of 6 mL suspension plus 54 mL diet. After vortexing, this mixture is poured into plastic trays with compartmentalized 3-ml wells (Nutrend Container Corporation, Jacksonville, Fla.). A water blank containing no control compound serves as the control. First instar larvae (USDA-ARS, Stoneville, Miss.) are placed onto the diet mixture. Wells are then sealed with Mylar sheeting (ClearLam Packaging, IL) using a tacking iron, and several pinholes are made in each well to provide gas exchange. Larvae were held at 25° C. for 6 days in a 14:10 (light:dark) holding room. Mortality and stunting are recorded after six days.

Bioassay by the top load method utilizes the same sample and diet preparations as listed above. The samples are applied to the surface of the insect diet. In a specific embodiment, surface area can range from 0.3 to approximately 0.8 cm² depending on the tray size, 96 well tissue culture plates were used in addition to the format listed above. Following application, samples are allowed to air dry before insect infestation. A water blank containing no control compound can serve as the control. Eggs are applied to each treated well and were then sealed with Mylar sheeting (ClearLam Packaging, IL) using a tacking iron, and pinholes are made in each well to provide gas exchange. Bioassays are held at 25° C. for 7 days in a 14:10 (light:dark) or 28° C. for 4 days in a 14:10 (light:dark) holding room. Mortality and insect stunting are recorded at the end of each bioassay.

Another assay useful according to the subject invention is the Western corn rootworm assay. Samples can be bioassayed against neonate western corn rootworm larvae (Diabrotica virgifera virgifera) via top-loading of sample onto an agar-based artificial diet at a rate of 160 ml/cm². Artificial diet can be dispensed into 0.78 cm² wells in 48-well tissue culture or similar plates and allowed to harden. After the diet solidifies, samples are dispensed by pipette onto the diet surface. Excess liquid is then evaporated from the surface prior to transferring approximately three neonate larvae per well onto the diet surface by camel's hair brush. To prevent insect escape while allowing gas exchange, wells are heat-sealed with 2-mil punched polyester film with 27HT adhesive (Oliver Products Company, Grand Rapids, Mich.). Bioassays are held in darkness at 25° C., and mortality scored after four days.

Analogous bioassays can be performed by those skilled in the art to assess activity against other pests, such as the black cutworm (Agrotis epsilon).

### EXAMPLE 6

### Target Pests

Toxins of the subject invention can be used, alone or in combination with other toxins, to control one or more non-mammalian pests. These pests may be, for example, those listed in Table 2. Activity can readily be confirmed using the bioassays provided herein, adaptations of these bioassays, and/or other bioassays well known to those skilled in the art.

TABLE 2
Target pest species

| ORDER/Common Name | Latin Name |
|---|---|
| LEPIDOPTERA | |
| European Corn Borer | Ostrinia nubilalis |
| European Corn Borer resistant to CrylA | Ostrinia nubilalis |
| Black Cutworm | Agrotis ipsilon |
| Fall Armyworm | Spodoptera frugiperda |
| Southwestern Corn Borer | Diatraea grandiosella |
| Corn Earworm/Bollworm | Helicoverpa zea |
| Tobacco Budworm | Heliothis virescens |
| Tobacco Budworm Rs | Heliothis virescens |
| Sunflower Head Moth | Homeosoma ellectellum |
| Banded Sunflower Moth | Cochylis hospes |
| Argentine Looper | Rachiplusia nu |
| Spilosoma | Spilosoma virginica |
| Bertha Armyworm | Mamestra configurata |
| Diamondback Moth | Plutella xylostells |
| COLEOPTERA | |
| Red Sunflower Seed Weevil | Smicronyx fulvus |
| Sunflower Stem Weevil | Cylindrocopturus adspersus |
| Sunflower Beetle | Zygoramma exclamationis |
| Canola Flea Beetle | Phyllotreta cruciferae |
| Western Corn Rootworm | Diabrotica virgifera virgifera |
| DIPTERA | Mayetiola destructor |
| Hessian Fly | |
| HOMOPTERA | Schizaphis graminum |
| Greenbug | |
| HEMIPTERA | Lygus lineolaris |
| Lygus Bug | |
| NEMATODA | Heterodera glycines |

### EXAMPLE 7

### Insertion of Toxin Genes Into Plants

One aspect of the subject invention is the transformation of plants with genes encoding the insecticidal toxin of the present invention. The transformed plants are resistant to attack by the target pest.

Genes encoding pesticidal toxins, as disclosed herein, can be inserted into plant cells using a variety of techniques which are well known in the art. For example, a large number of cloning vectors comprising a replication system in E. coli and a marker that permits selection of the transformed cells are available for preparation for the insertion of foreign genes into higher plants. The vectors comprise, for example, pBR322, pUC series, M13mp series, pACYC184, etc. Accordingly, the sequence encoding the Bacillus toxin can be inserted into the vector at a suitable restriction site. The resulting plasmid is used for transformation into E. coli. The E. coli cells are cultivated in a suitable nutrient medium, then harvested and lysed. The plasmid is recovered. Sequence analysis, restriction analysis, electrophoresis, and other biochemical-molecular biological methods are generally carried out as methods of analysis. After each manipulation, the DNA sequence used can be cleaved and joined to the next DNA sequence. Each plasmid sequence can be cloned in the same or other plasmids. Depending on the method of inserting desired genes into the plant, other DNA sequences may be necessary. If, for example, the Ti or Ri plasmid is used for the transformation of the plant cell, then at least the right border, but often the right and the left border of the Ti or Ri plasmid T-DNA, has to be joined as the flanking region of the genes to be inserted.

The use of T-DNA for the transformation of plant cells has been intensively researched and sufficiently described in EP 120 516; Hoekema (1985) In: The Binary Plant Vector System, Offset-durkkerij Kanters B. V., Alblasserdam, Chapter 5; Fraley et al., Crit. Rev. Plant Sci. 4:1-46; and An et al. (1985) EMBO J 4:277-287.

Once the inserted DNA has been integrated in the genome, it is relatively stable there and, as a rule, does not come out again. It normally contains a selection marker that confers on the transformed plant cells resistance to a biocide or an antibiotic, such as kanamycin, G 418, bleomycin, hygromycin, or chloramphenicol, inter alia. The individually employed marker should accordingly permit the selection of transformed cells rather than cells that do not contain the inserted DNA.

A large number of techniques are available for inserting DNA into a plant host cell. Those techniques include transformation with T-DNA using Agrobacterium tumefaciens or Agrobacterium rhizogenes as transformation agent, fusion, injection, biolistics (microparticle bombardment), or electroporation as well as other possible methods. If Agrobacteria are used for the transformation, the DNA to be inserted has to be cloned into special plasmids, namely either into an intermediate vector or into a binary vector. The intermediate vectors can be integrated into the Ti or Ri plasmid by homologous recombination owing to sequences that are homologous to sequences in the T-DNA. The Ti or Ri plasmid also comprises the vir region necessary for the transfer of the T-DNA. Intermediate vectors cannot replicate themselves in Agrobacteria. The intermediate vector can be transferred into Agrobacterium tumefaciens by means of a helper plasmid (conjugation). Binary vectors can replicate themselves both in E. coli and in Agrobacteria. They comprise a selection marker gene and a linker or polylinker which are framed by the right and left T-DNA border regions. They can be transformed directly into Agrobacteria (Holsters et al. Mol. Gen. Genet. 163:181-187). The Agrobacterium used as host cell is to comprise a plasmid carrying a vir region. The vir region is necessary for the transfer of the T-DNA into the plant cell. Additional T-DNA may be contained. The bacterium so transformed is used for the transformation of plant cells. Plant explants can advantageously be cultivated with Agrobacterium tumefaciens or Agrobacterium rhizogenes for the transfer of the DNA into the plant cell. Whole plants can then be regenerated from the infected plant material (for example, pieces of leaf, segments of stalk, roots, but also protoplasts or suspension-cultivated cells) in a suitable medium, which may contain antibiotics or biocides for selection. The plants so obtained can then be tested for the presence of the inserted DNA. No special demands are made of the plasmids in the case of injection and electroporation. It is possible to use ordinary plasmids, such as, for example, pUC derivatives. In biolistic transformation, plasmid DNA or linear DNA can be employed.

The transformed cells are regenerated into morphologically normal plants in the usual manner. If a transformation event involves a germ line cell, then the inserted DNA and corresponding phenotypic trait(s) will be transmitted to progeny plants. Such plants can be grown in the normal manner and crossed with plants that have the same transformed hereditary factors or other hereditary factors. The resulting hybrid individuals have the corresponding phenotypic properties.

In a preferred embodiment of the subject invention, plants will be transformed with genes wherein the codon usage has been optimized for plants. See, for example, U.S. Pat. No. 5,380,831. Also, advantageously, plants encoding a truncated toxin will be used. The truncated toxin typically will encode about 55% to about 80% of the full length toxin. Methods for creating synthetic Bacillus genes for use in plants are known in the art.

It should be understood that the examples and embodiments described herein are for illustrative purposes only and that various modifications or changes in light thereof will be suggested to persons skilled in the art and are to be included within the spirit and purview of this application and the scope of the appended claims.

1. An isolated TMOF receptor.

2. The isolated TMOF receptor, according to claim 1, wherein said receptor is from an insect.

3. The isolated TMOF receptor, according to claim 2, wherein said receptor is from a mosquito.

4. The isolated receptor, according to claim 1, wherein said receptor comprises the amino acid sequence shown in SEQ ID NO. 2.

5. An isolated polynucleotide encoding a TMOF receptor.

6. The isolated polynucleotide, according to claim 5, wherein said TMOF receptor is from an insect.

7. The isolated polynucleotide, according to claim 6, wherein said insect is a mosquito.

8. The isolated polynucleotide, according to claim 5, wherein said polynucleotide encodes a TMOF receptor which comprises the amino acid sequence shown in SEQ ID NO 2.

9. The isolated polynucleotide, according to claim 5, wherein said polynucleotide comprises the sequence shown in SEQ ID NO. 1.

10. The isolated polynucleotide, according to claim 5, wherein said polynucleotide hybridizes with the complement of SEQ ID NO. 2.

11. The isolated polynucleotide, according to claim 5, wherein said polynucleotide is optimized for expression in plants.

12. A compound which binds to a TMOF receptor, wherein said compound is not TMOF.

13. The compound, according to claim 12, wherein said receptor is from an insect.

14. The compound, according to claim 12, wherein said insect is a mosquito.

15. The compound, according to claim 12, wherein said receptor comprises the amino acid sequence shown in SEQ ID NO. 2.

16. The compound, according to claim 12, which is pesticidal.

17. The compound, according to claim 16, wherein said pest is an arthropod, a platyhelminth, or a nematode.

18. The compound, according to claim 17, wherein said pest is an insect.

19. The compound, according to claim 12, wherein said pest is selected from the group consisting of coleopterans, lepidopterans, and dipterans.

20. The compound, according to claim 19, wherein said pest is a mosquito.

21. A method for controlling a pest which comprises administering to said pest an effective amount of a compound which binds to a TMOF receptor, wherein said compound is not TMOF.

22. The method, according to claim 21, wherein said insect is selected from the group consisting of coleopterans, lepidopterans, and dipterans.

23. The method, according to claim 22, wherein said insect is a mosquito.

24. The method, according to claim 21, wherein said receptor comprises the amino acid sequence shown in SEQ ID NO 2.

25. A method for identifying an insecticidal compound wherein said method comprises determining if said compound binds to a TMOF receptor.

26. The method, according to claim 25, wherein said receptor is expressed at the surface of a cell.

27. The method, according to claim 25, wherein said TMOF receptor is from an insect.

28. The method, according to claim 27, wherein said insect is a mosquito.

29. The method, according to claim 25, wherein said receptor comprises SEQ ID NO. 2.

30. A method of screening trypsin synthesis-inhibiting compounds comprising exposing a compound in a competitive binding assay to a TMOF-receptor encoded by a nucleotide sequence comprising SEQ ID NO. 1.

31. The method, according to claim 30, wherein said TMOF-receptor is produced by a recombinant host transformed with said nucleotide sequence, and wherein said compound is exposed to said recombinant host.

32. The method, according to claim 31, wherein said recombinant host is a cell, and wherein said nucleotide sequence is expressed at the cell surface.

33. The method, according to claim 31, wherein said recombinant host is a cell, and wherein said TMOF receptor is secreted from the cell.

34. A DNA probe encoding an amino acid sequence shown in SEQ ID NO. 2, said probe having at least ten nucleotide residues.

35. An RNA molecule comprising a nucleotide sequence which is complimentary to a DNA sequence show in SEQ ID NO. 1, wherein said RNA molecule comprises at least 10 nucleotide residues.

36. An RNA molecule comprising a nucleotide sequence which is complimentary to a DNA sequence encoding an amino sequence shown in SEQ ID NO. 2, wherein said RNA molecule comprises at least 10 nucleotide residues.

37. A recombinant host transformed with a polynucleotide encoding a polypeptide exhibiting TMOF receptor.

38. The recombinant host, according to claim 37, wherein said polypeptide comprises the amino acid sequence shown in SEQ ID NO. 2.


SEQUENCE LISTING (2 sequences)

SEQ ID NO: 1 - DNA, 378 bp, Aedes aegypti
  CDS (1)..(189)
atactggggaggggggggggggacattgggttactcagttcagaccaaaggagtttcagcactgaaactctgcttaaagaactaaaaagagaagcggcggctgaggagcggagtgctgcctccaactcggggtcggtggttcccctctcggagcaaaggctgatgggacatctggcggccgcgctgtgagccggctttcctgctgccactttgggcgccttggatggagatcccaattgcagtttgtattttatttttttataagggacacgtggaaaaaccaaaccaaaccaaacaaagccaacaaaccacgacggtccttattttaaacctcagactccataaagaaacctttctatccaaaaaaaaaaaaaaaaa

SEQ ID NO: 2 - protein, 62 aa, Aedes aegypti
ILGRGGGDIGLLSSDQRSFSTETLLKELKREAAAEERSAASNSGSVVPLSEQRLMGHLAAAL